(19) United States
(12) Patent Application Publication
(10) Pub. No.: US 2005/0042710 A1
(43) Pub. Date: Feb. 24, 2005

(54) Sterility sampling test method and apparatus

(76) Inventors: Yasusuke Oshima, Ishikawa-Ken (JP); James E. Akers JR., Kansas City, MO (US)

Correspondence Address: FLYNN THIEL BOUTELL & TANIS, P.C., 2026 RAMBLING ROAD, KALAMAZOO, MI, 49008-1699, US

(21) Appl. No.: 10/918,951

(22) Filed: Aug. 16, 2004

(30) Foreign Application Priority Data
Aug. 22, 2003 (JP) 299015/2003

(51) Int. Cl. C12Q 1/22

(52) U.S. Cl. 435/31

(57) ABSTRACT

In a work isolator which is a work chamber maintained in a sterile condition, a predetermined sample containers to be subjected to a sterile test are extracted by a rejection device from containers filled with a material and sealed by a filling and sealing line, and a robot places the extracted sample containers on a collection pallet. The sample containers are moved from the work isolator to an accommodation device together with the pallet. The accommodation device is separated from the work isolator while being maintained in a sterile condition and is then connected to a test isolator which is a testing chamber maintained in a sterile condition. The sample containers are moved from the accommodation device into the test isolator while being maintained in a sterile condition. The sample containers are maintained in the sterile condition at all times from the work chamber to the testing chamber. Therefore, the need for sterilization of the outer surfaces of the sample containers at the time of sterility test in the testing chamber is eliminated and the drug sterility test can be immediately started.

### BACKGROUND OF THE INVENTION

1. Field of the Invention

The present invention relates to a sterility sampling test method and apparatus and, more particularly, to a sterility sampling test method and apparatus for testing sterility of a material filling a container in a testing chamber.

2. Description of the Prior Art

Conventionally, a process for production of a drug or the like includes means for maintaining sterility in a container such that a filling means for filling a material into the container and a sealing means for sealing the container are provided in a work chamber the interior of which is maintained in a sterile condition, and the container is sealed in a sterile condition by the sealing means.

To check whether the interior of the container is actually in a sterile condition, a sterility test is carried out in such a manner that a testing chamber the interior of which is maintained in a sterile condition is provided at a distance from the work chamber; several ones of containers sealed in the work chamber are extracted as sample containers; the sample containers are transported to the testing chamber; and the sterility of a material filling the sample containers is checked in the testing chamber.

As a device for extracting samples from containers filled with a material by filling means and which are sealed by sealing means for sealing the containers, one described in patent document 1 (Japanese Patent Laid-Open No. 2002-362502) is known.

According to the conventional art, as described above, the work chamber and the testing chamber are installed at a distance from each other, and a predetermined number of samples are extracted from containers carried out of the work chamber in a non-sterile condition and are transported into the testing chamber to be subjected to a sterility test in the testing chamber.

There is, therefore, a possibility of microorganisms being attached to the outer surfaces of the sample containers to affect testing results. For this reason, it is necessary to sterilize the outer surfaces of the sample containers in order to enable sterility testing. In fact, there has been a need to perform a time- and labor-consuming operation for sterilization before the sterility test.

### SUMMARY OF THE INVENTION

In view of the above-described problem, an object of the present invention is to provide a sterility sampling test method and apparatus which enable sampling for a sterility test to be performed with efficiency.

To achieve the above-described object, according to one aspect of the present invention, there is provided a sterility sampling test method in which a work chamber the interior of which is maintained in a sterile condition, sealing means of sealing containers in the work chamber and a testing chamber which is installed at a distance from the work chamber and the interior of which is maintained in a sterile condition are provided, and containers sealed by the sealing means in the work chamber are transported to the testing chamber to be subjected to a sterility test in the testing chamber, the method including a step of providing accommodation means capable of being connected to and being disconnected from each of the work chamber and the testing chamber while being maintained in a sterile condition, and holding a predetermined number of sample containers in the sealed containers without conveying the sample containers out of the work chamber, a step of connecting the accommodation means to the work chamber, a step of moving the sample containers in the work chamber into the accommodation means, a step of disconnecting from the work chamber the accommodation means in which the sample containers are accommodated, a step of connecting the accommodation means to the testing chamber, a step of moving the sample containers in the accommodation means into the testing chamber, and a step of testing sterility of a material filling each sample container by opening the sample container in the testing chamber.

According to another aspect of the present invention, there is provided a sterility sampling test apparatus having a work chamber the interior of which is maintained in a sterile condition, sealing means of sealing containers in the work chamber, and a testing chamber which is installed at a distance from the work chamber and the interior of which is maintained in a sterile condition, containers sealed by the sealing means in the work chamber being transported to the testing chamber to be subjected to a sterility test in the testing chamber, the apparatus including accommodation means capable of being connected to and being disconnected from each of the work chamber and the testing chamber while being maintained in a sterile condition, and transport means provided in the work chamber, the transport means accommodating in the accommodation means the containers sealed by the sealing means, while the accommodation means is maintained in the state of being connected to the work chamber.

According to the first aspect of the present invention, the sample containers sealed in the work chamber are accommodated in the accommodation means while being maintained in a sterile condition, and the accommodation means is thereafter connected to the testing chamber while being maintained in a sterile condition. Therefore, the sampling containers in the accommodation means are transported into the testing chamber while being always prevented from being subjected to any non-sterile environment.

Consequently, the need for sterilization of the outer surface of each sample container at the time of sterility testing is eliminated and sampling for the sterility test can be performed with efficiency.

According to the second aspect of the present invention, the sample containers sealed in the work chamber are accommodated in the accommodation means while being maintained in a sterile condition, and the accommodation means is thereafter connected to the testing chamber while being maintained in a sterile condition. Therefore, the containers in the accommodation means are transported into the testing chamber while being always prevented from being subjected to any non-sterile environment.

Consequently, the need for sterilization of the outer surface of each sample container at the time of sterility testing is eliminated and sampling for the sterility test can be performed with efficiency.

### BRIEF DESCRIPTION OF THE DRAWINGS

FIG. 1 is a plan view of a work isolator and a test isolator according to an embodiment of the present invention; and

FIGS. 2(a) and 2(b) are diagrams showing connection means, FIG. 2(a) showing a state before the establishment of connection by the connection means, FIG. 2(b) showing a state after the establishment of connection.

### DETAILED DESCRIPTION OF THE EMBODIMENTS

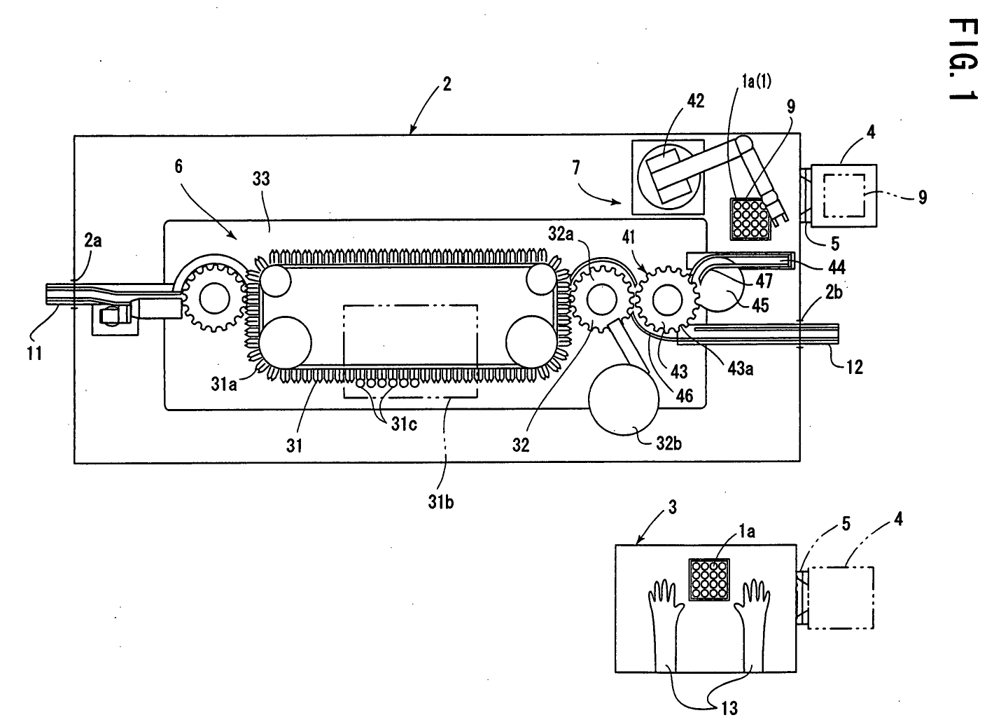

An embodiment of the present invention will be described with reference to the drawings. FIG. 1 shows a work isolator 2 which is a work chamber in which a container 1 is filled with a drug in the form of a liquid or a powder and is sealed, and a test isolator 3 which is a testing chamber in which a sterility test is performed on the drug in a sample container 1a extracted in the work isolator 2.

To the work isolator 2, an accommodation means 4 capable of accommodating a predetermined number of sample containers 1a is separably connected by a connection means 5. The accommodation means 4 can also be separably connected to the test isolator 3 by the connection means 5.

The work isolator 2 is provided with a filling and sealing line 6 for filling containers 1 with a drug and for sealing the containers 1, and a transport means 7 for taking out a predetermined number of sample containers 1a to be subjected to the sterility test from the containers 1 sealed in the filling and sealing line 6, and for transporting the sample containers 1a to the accommodation means 4.

A collection pallet 9 on which a predetermined number of sample containers 1a can be placed is provided in the vicinity of the transport means 7 and the accommodation means 4. Sample containers 1a taken out by the transport means 7 are transported temporarily onto the collection pallet 9 and are thereafter transported together with the collection pallet 9 to the accommodation means 4.

The above-described filling and sealing line 6 and transport means 7 are respectively controlled by a controller (not shown).

The interior of the isolator 2 is isolated from the outside atmosphere, is maintained in a sterile condition by means of sterilized air supplied from a sterile air supplier (not shown) and is maintained at a predetermined positive pressure.

Openings 2a and 2b are respectively formed in wall portions of the work isolator 2 on the left and right sides as viewed in FIG. 1. In the opening 2a, a first conveyor 11 for conveying containers 1 into the work isolator 2 is provided as a container 1 inlet. In the opening 2b, a second conveyor 12 for conveying containers 1 out of the work isolator 2 is provided as a container 1 outlet.

Air in the atmosphere in the work isolator 2 flows out through the openings 2a and 2b to prevent air in the outside atmosphere from flowing into the work isolator 2.

There is a need to sterilize the interior of the work isolator 2 before filling with a drug and sealing in the filling and sealing line 6. Therefore, a hydrogen peroxide solution sterilizer (not shown) is connected to the work isolator 2. The work isolator 2 is filled with hydrogen peroxide vapor supplied from the hydrogen peroxide solution to sterilize the interior of the work isolator 2.

The test isolator 3 is installed at a distance from the work isolator 2. For example, the work isolator 2 is installed in a clean room in a drug manufacturing factory, while the test isolator 3 is installed in an analysis room or the like on a floor of the drug manufacturing factory different from the floor on which the clean room exists.

The test isolator 3 is provided with gloves 13 in which the arms of an operator are inserted from the outside to perform operations necessary for the sterility test in the test isolator 3. The interior of the test isolator 3 is also maintained in a sterile condition by sterile air supplied from a sterilized air supplier (not shown) and is maintained at a predetermined positive pressure, as is that of the work isolator 2.

There is also a need to sterilize the interior of the test isolator before the sterility test. Therefore, sterilization in the test isolator 3 is also performed with a hydrogen peroxide solution sterilizer (not shown).

The accommodation means 4 is a movable container in which the above-described collection pallet 9 can be accommodated. When the accommodation means 4 is separated from the work isolator 2 or the test isolator 3, the interior of the accommodation means 4 is isolated from the outside atmosphere by the connection means 5 described below.

Therefore, there is no possibility of any microorganism entering the accommodation means 4 to be attached to the sample containers 1a when the accommodation means 4 is moved from the work isolator 2 to the test isolator 3.

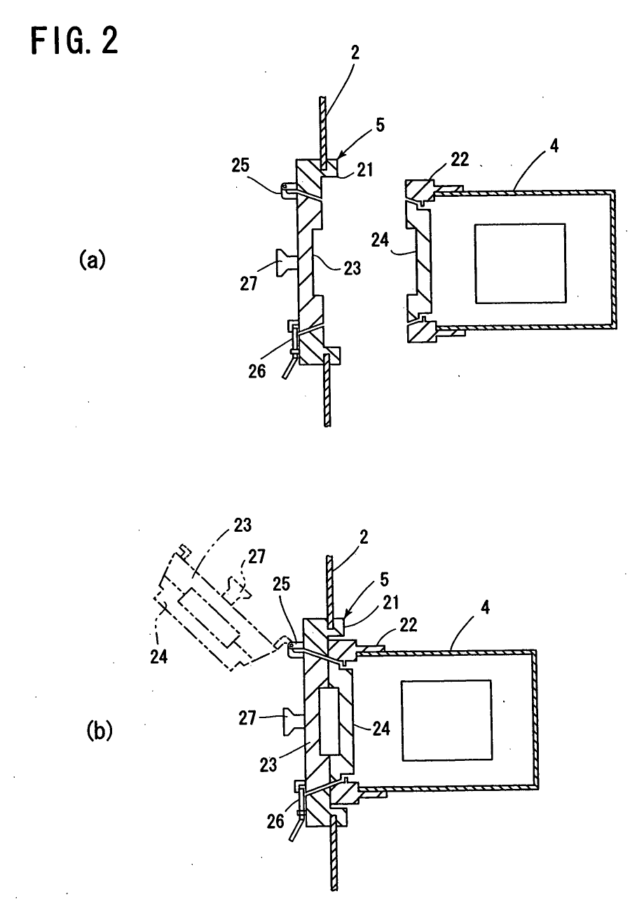

Description will be made of the connection means 5 with reference to FIGS. 2(a) and 2(b). The connection means 5 for connection to the work isolator 2 and the connection means 5 for connection to the test isolator 3 are identical in structure to each other. Description will therefore be made only of the connection means 5 for connection between the accommodation means 4 and the work isolator 2.

Connection rings 21 and 22 through which the above-described collection pallet 9 can be passed are respectively provided on the work isolator 2 and the accommodation means 4, and openable/closable partition members 23 and 24 are respectively provided in the connection rings 21 and 22.

A hinge 25 for axially supporting the partition member 23 is provided at an upper position on the connection ring 21 as viewed in FIG. 2(a), while a locking member 26 for locking the partition member 23 is provided at a lower position on the connection ring 21 as viewed in FIG. 2(a). The partition member 23 can be released from the locked state by rotating the locking member 26.

A knob 27 for opening/closing the partition member 23 is provided on the partition member 23 at a center of the same and on the work isolator 2 interior side.

The connection rings 21 and 22 are connected to each other as shown in FIG. 2(b). When the connection rings 21 and 22 are connected, air in the outside atmosphere does not flow into each of the work isolator 2 and the interior of the accommodation means 4.

When the connection rings 21 and 22 are connected to each other, the partition members 23 and 24 are simultaneously connected to each other. The partition member 24 is thereby made openable by rotating on the hinge 25 integrally with the partition member 23 toward into the work isolator 2.

In the above-described connection means 5, the portions of the connection rings 21 and 22 and partition members 23 and 24 exposed to the outside before the work isolator 2 and the accommodation means 4 are connected are not exposed to the atmosphere in the work isolator 2 when the work isolator 2 and the accommodation means 4 are connected. Therefore, there is no risk of contamination in the work isolator 2.

The construction of the connection means 5 described above is well known and is substantially the same as that described in Japanese Patent Laid-Open No. 6-193323. Therefore, no further description will be made of the construction of the connection means 5.

The above-described filling and sealing line 6 is constituted by a filling means 31 for filling containers 1 with a drug and sealing means 32 for sealing containers 1 filled with the drug. The filling and sealing line 6 is installed on a worktable 33 provided in the work isolator 2.

The portions of the filling means 31 and the sealing means 32 above the worktable 33 are exposed to the atmosphere in the work isolator 2, while drive means (not shown) for driving the driving the filling means 31 and the sealing means 32 are provided below the worktable 33. Dust particles generated from the drive means are thereby prevented from entering the work isolator 2.

The filling means 31 is constituted by grippers 31a which receive and convey containers 1 conveyed by the first container 11, and a filling mechanism 31b for filling containers 1 with a drug. A plurality of filling nozzles 31c are provided in the filling mechanism 31b so as to be able to reciprocate to fill containers 1 with a drug while following the movement of the containers 1 conveyed by the grippers 31a.

The sealing means 32 includes a rotary capping wheel 32a which receives containers 1 one by one from the grippers 31a and conveys received containers 1, a capping head (not shown) which seals containers 1 conveyed by the capping wheel 32a, and a plug feeder 32b which supplies plugs to the capping head. Containers 1 sealed by the sealing means 32 are conveyed to the subsequent process step by the second conveyor 12.

No further description will be made of the above-described filling means 31 and sealing means 32 because they are well known.

The transport means 7 includes a rejection means 41 for extracting sample containers 1a to be subjected to the sterility test from containers 1 sealed by the sealing means 32, and a robot 42 which is a moving and placing means for accommodating in the accommodation means 4 sample containers 1a extracted by the rejection means 41. Drive means (not shown) for driving the rejection means 41 and the robot 42 are also provided below the worktable 33.

The rejection means 41 is provided between the capping wheel 32a and the second conveyor. The rejection means 41 includes a rejection wheel 43 which receives sample containers 1a from the capping wheel 32a, and a turn table 45 which conveys sample containers 1a received by the rejection wheel 43 to a third conveyor 44 at an extraction position.

The rejection wheel 43 rotates anticlockwise as viewed in FIG. 1. A guide 46 is formed along the circumference of the rejection wheel 43 so as to extend from the capping wheel 32a to the second conveyor 12. Sample containers 1a received by the rejection wheel 43 from the capping wheel 32a are conveyed onto the second conveyor 12 along the guide 46.

Pockets 43a in each of which one sample container 1a is accommodated are formed in the rejection wheel 43 at the circumference of the same. A negative pressure is produced in each pocket 43a by a negative pressure source (not shown) This negative pressure can be freely changed by the above-mentioned controller.

When the controller produces a negative pressure in a predetermined one of the pockets 43a, sample container 1a is sucked into the pocket 43a in which the negative pressure is produced to be conveyed to the turn table 45 while passing by the second conveyor 12.

A guide 47 having a width corresponding to that of sample containers 1a is formed so as to extend from the turn table 45 to the third conveyor 44. When sample container 1a sucked to the rejection wheel 43 is released from the pocket 43a at an upstream position on the guide 47, it moves from the turn table 45 onto the third conveyor 44 along the guide 47.

A well-known conventional industrial robot is used as the above-mentioned robot 42. However, the portion of the robot 42 exposed in the work isolator 2 is covered with a material not corroded by hydrogen peroxide to enable sterilization of the surface of the robot 42 as well as sterilization of the interior of the work isolator 2 with hydrogen peroxide vapor.

The robot 42 can move sample containers 1a on the third conveyor 44 to the collection pallet 9. When a predetermined number of sample containers 1a are placed on the collection pallet 9, the robot 42 can move the sample containers 1a into the accommodation means 4 together with the collection pallet 9.

The robot 42 can also open or close the partition members 23 and 24 of the connection means 5 by operating the locking member 26 and the knob 27 of the connection means 5.

For a sterilization test of containers 1 filled and sealed in the work isolator 2 by the process based on the above-described arrangement in this embodiment, sample containers 1a are sampled as described below.

The accommodation means 4 the interior of which is sterilized in advance is connected to the work isolator 2. Also, the interior of each of the work isolator 2 and the test isolator 3 is sterilized with hydrogen peroxide vapor.

When the operation of the filling and sealing line 6 in the work isolator 2 is started, sterilized containers 1 are conveyed by the first conveyor 11 and are moved to a position below the filling nozzles 31c by the grippers 31a of the filling means 31.

Each filling nozzle 31c having its end inserted in one container 1 fills the container 1 with a predetermined amount of a drug while moving by following the container 1. After the completion of filling, the nozzle end is pulled out from the interior of the container 1.

The container 1 filled with the drug is moved from the gripper 31a to the capping wheel 32a of the sealing means 32, and the capping head (not shown) seals the container 1 with a plug supplied from the plug feeder 32b.

The container 1 sealed at the capping wheel 32a is received from the capping wheel 32 by the pocket 43a of the rejection wheel 43 to be accommodated in the pocket 43a.

If the container 1 accommodated in the pocket 43a is immediately conveyed as a product to the subsequent process step, the controller does not produce any negative pressure in the pocket 43a in which the container 1 is accommodated. The container 1 released from the pocket 43a at the upstream position on the second conveyor 12 and is conveyed out of the work isolator 2 through the opening 2b by the second conveyor 12.

If the container 1 accommodated in the pocket 43a is sampled as sample container 1a, the controller produces a negative pressure in the pocket 34a in which the sample container 1a is accommodated. The sample container 1a is thereby sucked into the pocket 34a.

When the sample container 1a reaches the guide 47 with the rotation of the rejection wheel 43, the controller removes the negative pressure to release the sample container 1a. The released sample container 1a is conveyed on the turn table 45 and the third conveyer and temporarily held in a downstream portion of the third conveyor 44.

When the number of sample containers 1a that have reached the third conveyor 44 becomes equal to the predetermined number, the robot 42 is operated to hold the sample containers 1a and place the sample containers 1a on the collection pallet 9.

While in this embodiment sixteen sample containers 1a are placed on the collection pallet 9, the number of sample containers 1a to be extracted and the timing of extraction can be freely set. For example, sample containers 1a may be extracted mainly at about a time immediately after a start of the filling operation, may be extracted one by one at predetermined time intervals, or may be extracted at times randomly set by using random numbers.

After the completion of the operation in the work isolator 2 or during the operation, the robot 42 operates the locking member 26 of the connection means 5 to disengage this member and opens the partition members 23 and 24 by holding the knob 27. The robot 42 then moves sample containers 1a into the accommodation means 4 together with the collection pallet 9 by holding the collection pallet 9.

After moving the collection pallet 9, the robot 42 closes the partition members 23 and 24 by again holding the knob 27, and locks the partition members 23 and 24 by operating the locking member 26.

The accommodation means 4 is then manually separated from the work isolator 2, is transported to the analysis room in which the test isolator 3 is installed, and is connected to the test isolator 3 installed in the analysis room by using the connection means 5.

At this time, the accommodation means 4 is transported in a non-sterile condition. However, the accommodation means 4 is isolated from the outside atmosphere by the partition member 24 and there is, therefore, no possibility of any foreign material or microorganism being attached to the outer surfaces of sample containers 1a.

The accommodation means 4 may be transported by an operator or may be transported by human power in a state of being placed on a truck or the like. Further, wheels may be attached to the accommodation means 4.

When the accommodation means 4 is connected to the test isolator 3, an operator equips him/herself with the gloves 13, opens the partition members 23 and 24 by operating the locking member 26 and the knob 27 of the connection means 5, moves the sample containers 1a into the test isolator 3 together with the collection pallet 9, and closes the partition members 23 and 24 again.

Thereafter, the operator opens each sample container 1a in the test isolator 3, takes out the material filling the sample container, subjects the material to cultivation for a predetermined time period, and checks whether or not a microorganism has proliferated, thus making a sterility test.

As described above, sample containers 1a are maintained in a sterile condition at all times from the work isolator 2 to the test isolator 3. Therefore, there is no need to sterilize the outer surfaces of the sample containers 1a in the test isolator 3 at the time of sterilization testing as in the case of the conventional art, and thus a drug sterility test can be immediately started.

The above-described container 1 may be, for example, a resin bottle, a vial, an ampule, or a syringe. The sealing means 32 may seal the container 1 by a corresponding means, i.e., a resin cap, a rubber plug, aluminum sealing, weld sealing, stopper plugging or the like. The construction of the sealing means 32 is not limited to the above-described one constituted by the capping wheel 32 and so on. As the sealing means 32, a well-known conventional sealing means corresponding to the sealing method may be used.

Also, the construction of the rejection means 41 is not limited to the above-described one using the rejection wheel 43. Any of well-known arrangements capable of extracting conveyed containers 1 may be adopted.

For example, the arrangement may be such that the robot 42 is operated so as to follow the movement of containers 1, extract containers 1a and place the containers 1a on the collection pallet 9. If the robot 42 is used in this manner, the rejection means 41 can be removed. Further, sample containers 1a held by the robot 42 may be directly accommodated in the accommodation means 4 without being placed on the collection pallet 9.

The arrangement may also be such that gloves 13 similar to those in the test isolator 3 are provided instead of the robot 42 in the vicinity of the rejection means 41 in the isolator 2, an operator manually moves sample containers 1a from the rejection means 41 onto the collection pallet 9 by using the gloves 13, and the collection pallet 9 is thereafter moved into the accommodation means 4.

1. A sterility sampling test method in which a work chamber the interior of which is maintained in a sterile condition, sealing means of sealing containers in the work chamber and a testing chamber which is installed at a distance from the work chamber and the interior of which is maintained in a sterile condition are provided, and containers sealed by the sealing means in the work chamber are transported to the testing chamber to be subjected to a sterility test in the testing chamber, said method comprising:
a step of providing accommodation means capable of being connected to and being disconnected from each of the work chamber and the testing chamber while being maintained in a sterile condition, and holding a predetermined number of sample containers in the sealed containers without conveying the sample containers out of the work chamber;
a step of connecting the accommodation means to the work chamber;
a step of moving the sample containers in the work chamber into the accommodation means;
a step of disconnecting from the work chamber the accommodation means in which the sample containers are accommodated;
a step of connecting the accommodation means to the testing chamber;
a step of moving the sample containers in the accommodation means into the testing chamber; and
a step of testing sterility of a material filling each sample container by opening the sample container in the testing chamber.

2. A sterility sampling test apparatus having a work chamber the interior of which is maintained in a sterile condition, sealing means of sealing containers in the work chamber, and a testing chamber which is installed at a distance from the work chamber and the interior of which is maintained in a sterile condition, containers sealed by the sealing means in the work chamber being transported to the testing chamber to be subjected to a sterility test in the testing chamber, said apparatus comprising:
accommodation means capable of being connected to and being disconnected from each of the work chamber and the testing chamber while being maintained in a sterile condition; and
transport means provided in the work chamber, the transport means accommodating in said accommodation means the containers sealed by the sealing means, while said accommodation means is being maintained in the state of being connected to the work chamber.

3. A sterility sampling test apparatus according to claim 2, wherein the work chamber has an inlet through which containers are conveyed from the outside into the work chamber while being maintained in a sterile condition, filling means of filling the containers with a material, and an outlet through which the containers filled with the material by said filling means and sealed by the sealing means are conveyed to the outside while being maintained in a sterile condition, and said transport means includes rejection means of moving a plurality of number of sample containers in the containers sealed by the sealing means to an extraction position set in the work chamber, and moving and placing means of enabling the sample containers at the extraction position to be accommodated in the accommodation means.
